(19) United States
(12) Patent Application Publication
(10) Pub. No.: US 2020/0404902 A1
(43) Pub. Date: Dec. 31, 2020

(54) SYSTEM FOR PASSIVE PERMEATION OF A BIOLOGICAL MATERIAL AND METHOD OF USING SAME

(71) Applicant: Cook Medical Technologies LLC, Bloomington, IN (US)

(72) Inventors: Erin Roberts, Bloomington, IN (US); Jorge L. Jimenez-Rios, Bloomington, IN (US); Victor W. Havill, Bloomington, IN (US)

(73) Assignee: Cook Medical Technologies LLC, Bloomington, IN (US)

(21) Appl. No.: 16/911,812

(22) Filed: Jun. 25, 2020

Related U.S. Application Data
(60) Provisional application No. 62/867,397, filed on Jun. 27, 2019.

(51) Int. Cl. A01N 1/02; C12M 1/00

(52) U.S. Cl. CPC A01N 1/0242; C12M 23/22; A01N 1/0221

(57) ABSTRACT

A system for passive permeation of a biological material is disclosed. The system may include a main channel extending between an upper portion and a lower portion; a main reservoir connected to the upper portion of the main channel and in fluid communication with the main channel; a bottom reservoir connected to the lower portion of the main channel and in fluid communication with the main channel; at least one secondary channel disposed in at least one position between the upper portion and the lower portion of the main channel such that fluid communication is established in the at least one position between the at least one secondary channel and the main channel; and at least one secondary reservoir respectively connected to an upper portion of each of the at least one secondary channel and in fluid communication with the respective at least one secondary channel.

### CROSS-REFERENCE TO RELATED APPLICATIONS

This application is a non-provisional application which claims priority to U.S. provisional application Ser. No. 62/867,397, filed Jun. 27, 2019, which is incorporated by reference herein in its entirety.

### TECHNICAL FIELD

In order to cryopreserve cells through vitrification, the cells must be permeated into vitrification solutions, consisting on media buffer with a high concentration of cryoprotectant agents that help the cell survive the vitrification process. The current procedure involves a highly skilled user manually aspirating and injecting the cells into various solutions of media, with increasing concentrations of cryoprotectant agents until the final concentration is reached. This procedure requires a significant amount of time by the user. The cells of interest can range in size from 50 to 200 microns, creating the need for the procedure to be performed under a microscope using micromanipulation pipettes operated by hand. Successful permeation of cells in the vitrification solutions requires careful timing and precision handling of micropipettes.

### BRIEF SUMMARY OF THE INVENTION

One general aspect of the present disclosure includes a system for passive permeation of a biological material, including a main channel extending between an upper portion and a lower portion; a main reservoir connected to the upper portion of the main channel and in fluid communication with the main channel; a bottom reservoir connected to the lower portion of the main channel and in fluid communication with the main channel; at least one secondary channel disposed in at least one position between the upper portion and the lower portion of the main channel such that fluid communication is established in the at least one position between the at least one secondary channel and the main channel; and at least one secondary reservoir respectively connected to an upper portion of each of the at least one secondary channel and in fluid communication with the respective at least one secondary channel.

Another general aspect of the present disclosure includes a system for cryopreservation of a biological material, including a main channel extending between an upper end and a bottom end. The main channel includes a first section disposed proximate to the upper end of the main channel and a second section disposed proximate to the bottom end of the main channel. The first section is filled with a first liquid having a first cryoprotectant concentration and the second section is filled with a second liquid having a second cryoprotectant concentration greater than the first cryoprotectant concentration. The main channel is configured such that a biological material placed into the main channel through the upper end migrates towards the bottom end by gravity, and when the biological material reaches the bottom end, the biological material is ready for vitrification.

Another general aspect of the present disclosure includes a method of passively permeating a biological material using a system having a main channel in fluid communication with at least one secondary channel, the main channel being connected to a main reservoir at an upper portion of the main channel and being connected to a bottom reservoir at a lower portion of the main channel, and the at least one secondary channel being connected to at least one secondary reservoir. The method includes placing a first predetermined amount of a main liquid in the main reservoir; placing at least one predetermined amount of at least one secondary liquid in the at least one secondary reservoir; allowing the main liquid to flow along the main channel; allowing the at least one secondary liquid to flow along the at least one secondary channel, and then into the main channel and mix with the main liquid to form at least one mixed liquid; and placing a biological material in the main reservoir such that the biological material migrates along the main channel by gravity to travel through the at least one mixed liquid and reaches the bottom reservoir.

Other systems, methods, features and advantages of the invention will be, or will become, apparent to one with skill in the art upon examination of the following figures and detailed description. It is intended that all such additional systems, methods, features, and advantages be within the scope of the invention, and be encompassed by the following claims.

### BRIEF DESCRIPTION OF THE DRAWINGS

The present disclosure can be better understood with reference to the following drawings and description. The components in the figures are not necessarily to scale, emphasis instead being placed upon illustrating the principles of the present disclosure. Moreover, in the figures, like-referenced numerals designate corresponding parts throughout the different views.

FIG. 1 is an illustration showing a side view of a system for passive permeation of a biological material in accordance with certain aspects of the present disclosure.

FIG. 2 is an illustration showing the system of FIG. 1 is incorporated into a permeation device in accordance with certain aspects of the present disclosure.

FIG. 3 is an illustration showing another embodiment of the system for passive permeation of a biological material in accordance with certain aspects of the present disclosure.

FIG. 4 is an illustration showing the system of FIG. 3 is connected to a petri dish that sits in a microscope in accordance with certain aspects of the present disclosure.

FIG. 5 is an illustration showing the concentration of liquids in a biological material as it flows through the system of FIG. 1 in accordance with certain aspects of the present disclosure.

### DETAILED DESCRIPTION

Various aspects are described below with reference to the drawings in which like elements generally are identified by like numerals. The relationship and functioning of the various elements of the aspects may better be understood by reference to the following detailed description. However, aspects are not limited to those illustrated in the drawings or explicitly described below. It also should be understood that the drawings are not necessarily to scale, and in certain instances details may have been omitted that are not necessary for an understanding of aspects disclosed herein, such as conventional material, construction, and assembly.

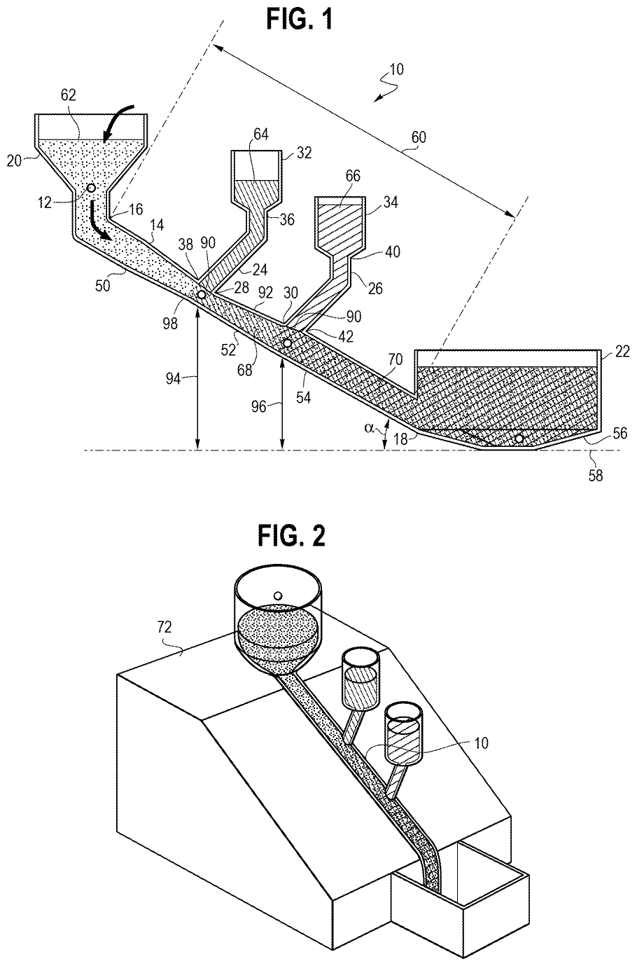

Referring to FIG. 1, a system 10 for passive permeation of a biological material is shown. The system may include a main channel 14 extending between an upper portion 16 and a lower portion 18. The main channel 14 may have a tubular configuration or any configuration suitable for a biological material and liquids to travel through. A main reservoir 20 may be connected to the upper portion 16 of the main channel 14 and in fluid communication with the main channel 14. A bottom reservoir 22 may be connected to the lower portion 18 of the main channel 14 and in fluid communication with the main channel 14. The system 10 may be configured such that when the bottom reservoir 22 is placed on a planar surface 58, the main channel 14 may extend from the lower portion 18 to the upper portion 16 at an angle α to the planar surface 58, which allows a main liquid 62 and a biological material 12 placed in the main reservoir 20 to migrate by gravity along the main channel 14 to the bottom reservoir 22. In some embodiments, the angle α may be in the range of about 10 degrees to about 60 degrees. The biological material 12 may be various kinds of biological materials, such as an embryo or an oocyte. The main channel 14 may be configured to accommodate the various kinds of biological materials. For example, the main channel 14 may be configured to accommodate a large blastocyst-stage embryo, that is, the main channel 14 may be larger than about 0.3 mm and generally up to about 3 mm in width. The term “about” is specifically defined herein to include the specific value referenced as well as a dimension that is within 5% of the dimension both above and below the dimension.

The system 10 may include one or more spaced-apart secondary channels that are connected to the main channel 14 and in fluid communication with the main channel 14. In some embodiments, as shown in FIG. 1, the system 10 may include a first secondary channel 24 and a second secondary channel 26. The first secondary channel 24 may extend from a lower portion 38 to an upper portion 36, where the lower portion 38 may be connected to the main channel 14 in a first position 28 such that the fluid communication between the main channel 14 and the first secondary channel 24 is established in the first position 28. The second secondary channel 26 may extend from a lower portion 42 to an upper portion 40, where the lower portion 42 may be connected to the main channel 14 in a second position 30 such that the fluid communication between the main channel 14 and the second secondary channel 26 is established in the second position 30. The first and second secondary channels 24, 26 may be spaced along a length 60 of the main channel 14, where the first secondary channel 24 is disposed closer to the upper portion 16 of the main channel 14 than the second secondary channel 26. In some embodiments, the vertical distance between a first outer surface 98 of the main channel 14 and the planar surface 58 may be between about 0.1 inch to about 9 inches. In some embodiments, a second vertical distance 96 between the first outer surface 98 of the main channel 14 at the second position 30 and the planar surface 58 may be about 30% to about 75% of a first vertical distance 94 between the first outer surface 98 of the main channel 14 at the first position 28 and the planar surface 58.

First and second secondary reservoirs 32, 34 may be respectively connected to the upper portions 36, 40 of the first and second secondary channels 24, 26 and in fluid communication with the respective first and second secondary channels 24, 26. The first and second secondary reservoirs 32, 34 may be configured to receive a first secondary liquid 64 and a second secondary liquid 66, respectively, and allow the first and second secondary liquids 64, 66 placed therein to flow, along the respective secondary channels 24, 26, into the main channel 14 and mix with the main liquid 62 flowing from the main reservoir 20, such that mixed liquids may be formed along the length 60 of the main channel 14. In some embodiments, after allowing the first secondary and the second secondary liquids 64, 66 to mix with the main liquid 62 for respective predetermined amount of time, first, second, and third sections 50, 52, 54 of the main channel 14 with the main liquid 62, a first mixed liquid 68, and a second mixed liquid 70 may be respectively established, along the length 60 of the main channel 14, between the main reservoir 20 and the first secondary channel 24 (e.g., the first position 28), between the first and second secondary channels 24 and 26 (e.g., between the first and second positions 28, 30), and between the second secondary channel 26 (e.g., the second position 30) and the bottom reservoir 22 (e.g., the bottom 56 of the bottom reservoir 22).

In use, a user may place predetermined amount of the main liquid 62, the first secondary liquid 64, and the second secondary liquid 66 in the main reservoir 20, the first secondary reservoir 32, and the second secondary reservoir 34 respectively. In some embodiments, the total amount of liquids used may be in the range of 1 to 10 ml total, depending on the configuration of the system 10 and the desired exposure time for the biological material 12. The fluid dynamics of the system 10 will create a flow of the main liquid 62 that is subsequently fed by the first and second secondary liquids 64, 66. After a predetermined amount of time, desired first and second mixed liquids 68, 70 may be formed in the second and third sections 52, 54 respectively.

Then, a user may place the biological material 12 in the main reservoir 20, such that as the biological material 12 migrates down the main channel 14 by gravity, the biological material 12 will be sequentially permeated with the main liquid 62, the first mixed liquid 68, and the second mixed liquid 70 for respective times of traveling from the main reservoir 20 to the first position 28, from the first position 28 to the second position 30, and from the second position 30 to the bottom 56 of the bottom reservoir 22. The system 10 may be configured such that the biological material 12 migrating along the main channel 14 sequentially travels through the first, second, and third sections 50, 52, and 54 for respective first, second, and third predetermined amount of time. The respective traveling times (i.e., permeation times) of the biological material 12 through the first, second, and third sections 50, 52, and 54 of the main channel 14 may be varied as needed and/or desired by varying the length 60 and diameter of each of the first, second, and third sections 50, 52, and 54, the angle α, and the concentrations of the main, first secondary, and second secondary liquids 62, 64 and 66, depending on the desired and/or needed permeation process of the biological material 12. One of ordinary skill in the art with a thorough review of this disclosure will be able to optimize the length, diameter, and angles of the components of the system with merely routine optimization and without undue experimentation.

In use, a user may observe the bottom reservoir 22 under a microscope to wait for the biological material 12 to arrive at the bottom reservoir 22. In some embodiments, the system 10 may be made of clear plastic that does not react to the liquids used with the system 10, such that a process of the biological material 12 migrating from the main reservoir 20 to the bottom reservoir 22 is visible to a user. In some embodiments, the system 10 may be configured such that the biological material 12 travels slowly enough for the user to follow it using the microscope as it travels down the main channel 14. In some embodiments, as shown in FIG. 2, the system 10 may be incorporated into a device 72, where the device 72 is manufactured of a clear plastic and the dimensions of the device 72 may be between about 1 and 3 inches in either direction.

The first and second secondary channels 24, 26 and the first and second secondary reservoirs 32, 34 may be configured (e.g., shape, length, dimension) such that the first and second secondary channels 24, 26 are always at higher pressure than the main channel 14 such that the biological material 12 migrates along the main channel 14 towards the bottom 56 of the bottom reservoir 22 without migrating towards the first and second secondary channels 24, 26. In some embodiments, the differences in pressure are achieved via a combination of height and channel length. The longer a channel is, the higher the pressure drop that the flow experiences, due to head loss through the channel (friction). In some embodiments, the main channel 14 may have a length between about 1 inch and about 12 inches, and the lengths of the first and second secondary channels 24, 26 each may be smaller than half of the length of the main channel 14. Using this, the main channel 14 may be configured such that the first and second secondary channels 24, 26 are at higher pressure than the main channel 14 so that the first and second secondary liquids 64, 66 merge into the main liquid 62. In the meantime, the elevation of the first and second secondary liquids 64, 66 in the first and second secondary reservoirs 32, 34 may be higher than the elevation of the main liquid 62 in the main reservoir 20 and the density of the first and second secondary liquids 64, 66 in the first and second secondary reservoirs 32, 34 may be greater than the density of the main liquid 62 in the main reservoir 20. In some embodiments, valves (e.g., one-way check-valve) may be provided at the lower portions 38, 42 of the respective first and second secondary channels 24, 26 to facilitate preventing the main liquid 62 and the biological material 12 from migrating towards the first and second secondary channels 24, 26.

The system 10 provides the ability to automate the passive permeation process of a biological material by using gravity, thereby reducing the amount of time needed to perform the permeation process, increasing accuracy of the process, and eliminating the need for careful timing and precision handling of micropipettes.

Although a system 10 with two secondary channels are specifically depicted and described above, it will be appreciated that the number of secondary channels may be varied as desired and/or needed, without departing from the scope of the present invention, to achieve a desired permeation process of the biological material 12. For example, a system 10 having a greater number of secondary channels may allow the biological material 12 placed in the main reservoir 20 to be permeated with a greater number of mixed liquids. As described above, the configuration and spacing of the two or more secondary channels may be varied as needed and/or desired depending on the respective desired permeation times in the main liquid 62 and the two or more mixed liquids.

In some embodiments, the system 10 may include a port 90 configured such that the biological material 12 can be flushed out of the system 10. In some embodiments, a second outer surface 92 of the system 10 may be removable such that the biological material 12 disposed in the system 10 can be manually retrieved by a user.

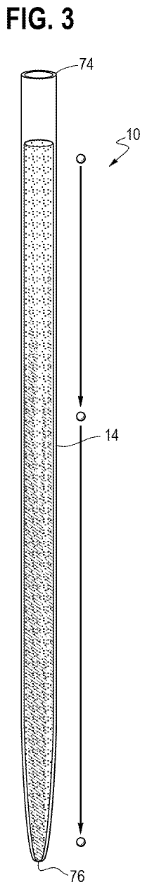

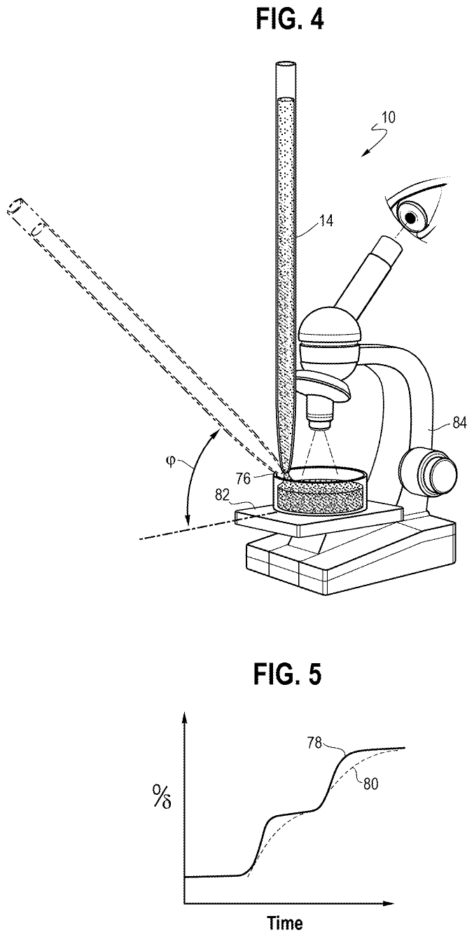

In some embodiments, as shown in FIG. 3, the system 10 may include a single main channel 14 extending between an upper end 74 to a bottom end 76. The single main channel 14 may include two or more sections with different liquids, such that a biological material 12 placed in the single main channel 14 through the upper end 74 may migrate down the single main channel 14 by gravity, thereby the biological material 12 is permeated with each of the different liquids for a predetermined amount of time, depending on the length of each section, to achieve a desired permeation process of the biological material 12. In some embodiments, as shown in FIG. 4, the bottom end 76 of the single main channel 14 may be connected to a petri dish that sits on a supporting surface 82 of a microscope 84. The angle φ of the single main channel 14 relative to the supporting surface 82 may be configured (up to fully vertical) such that desired traveling/permeation time of the biological material 12 through the single main channel 14 may be achieved.

In some embodiments, the system 10 may be used with cryoprotectant solutions for cryopreservation of a biological material such that the biological material is ready for vitrification. While a system 10 for passive permeation of an embryo for cryopreservation of the embryo is specifically described herein, the system 10 may be successfully implemented for use with other types of liquids and/or other types of biological materials (e.g., oocytes) for other medical and/or experimental uses. For the sake of brevity, a system disclosed herein is described and depicted as a system for cryopreservation of an embryo, one of ordinary skill in the art, with a thorough review of the subject specification and figures, would readily comprehend how the system may be implemented for convenient passive permeation of other types of biological materials with the same or other types of liquids for the same or other medical and/or experimental uses, and would comprehend which other types of biological materials, liquids, and uses might be suitable without undue experimentation.

When the system 10 with first and second secondary channels 24 and 26 is used for cryopreservation of an embryo 12, the system 10 may be provided with cryoprotectant solutions with different concentrations. In some embodiments, for example, cryoprotectant solutions with increasing cryoprotectant concentrations may be respectively placed in the main reservoir 20, the first secondary reservoir 32, and the second secondary reservoir 34. After allowing the first and the second secondary liquids 64, 66 to mix with the main liquid 62 for respective predetermined amount of time, first, second, and third sections 50, 52, and 54 of the main channel 14 with increasing cryoprotectant concentrations may be respectively established, along the length 60 of the main channel 14, between the main reservoir 20 and the first secondary channel 24, between the first and second secondary channels 24 and 26, and between the second secondary channel 26 and the bottom reservoir 22.

Then, a user may place an embryo 12 in the main reservoir 20, and the embryo 12 will migrate down the main channel 14 by gravity to be permeated with the cryoprotectant solutions with increasing concentrations. The system 10 may be configured such that the embryo 12 migrating along the main channel 14 sequentially travels through the first, second, and third sections 50, 52, and 54 for respective first, second, and third predetermined amount of time. The first, second, and third predetermined amount of time may be selected such that when the embryo 12 migrating from the main reservoir 20 reaches the bottom reservoir 22, the embryo 12 is ready for vitrification. In some embodiments, the system 10 is configured such that the embryo 12 may spend no more than 15 minutes in the system 10. For example, the system 10 may be configured such that the respective traveling time of the embryo 12 through each of the first, second, and third sections 50, 52, and 54 may range from 30 seconds to 5 minutes. In some other embodiments, the system 10 may be configured such that the respective traveling time of the embryo 12 through each of the first, second, and third sections 50, 52, and 54 may range from 30 seconds to 2 minutes. For example, the system 10 may be configured such that the embryo 12 may travel in the first section 50 for about 1 minute, and then travel in the second section 52 for about 2 minutes, and then travel in the third section 54 for about 20 seconds to about 30 seconds. When the embryo 12 reaches the bottom 56 of the bottom reservoir 22, the user may confirm the presence of the embryo 12 using a microscope. Then the user may extract the embryo 12 from the bottom 56 of the bottom reservoir 22 and places the embryo 12 in a device for vitrification.

As discussed above, each of the second and third sections 52 and 54 may have gradually increasing cryoprotectant concentration, such that the embryo 12 migrating down the main channel 14 may be permeated with gradually increasing cryoprotectant concentrations. The increasing pattern of the cryoprotectant concentrations in the second and third sections 52 and 54 may be varied as desired and/or needed by varying the configuration of the system 10 (e.g., the length 60 of main channel 14, the location of the first and second positions 28 and 30, the height of first and second secondary reservoirs 32 and 34, the diameter of the main channel 14 and the first and second secondary channels 24 and 26, and the cryoprotectant concentration of the first and second secondary liquids 64 and 66) such that desired and/or needed mixing rates of the main liquid 62 and the first and second secondary liquids 64 and 66 may be achieved. In some embodiments, for example, the system 10 is configured such that the achieved greatest cryoprotectant concentrations in the first, second, and third sections 50, 52, and 54 of the main channel 14 may be 0%, 17%, and 55% by volume respectively.

The increasing pattern of the cryoprotectant concentrations in the second and third sections 52 and 54 may determine how gradually the embryo 12 experiences the changes in cryoprotectant concentration. In some embodiments, as shown in FIG. 5, when the first and second secondary liquids 64, 66 mix with the main liquid 62 fast, the embryo 12 may experience two fast increases in cryoprotectant concentration (e.g., as shown as the curve 78). When first and second secondary liquids 64, 66 mix with the main liquid 62 slowly, the embryo 12 may experience two gradual increases in cryoprotectant concentration (e.g., as shown as the curve 80).

In some embodiments, after desired cryoprotectant concentrations are achieved in the first, second, and third sections 50, 52, and 54 of the main channel 14, respectively, a system for detaching the main channel 14 from the first and second secondary channels 24, 26 may be used, such that a single main channel 14 with desired concentration gradient already present may be formed (e.g., as shown in FIG. 3), in which the embryo 12 may travel down gradually increasing cryoprotectant concentrations. After the embryo 12 reaches the bottom end 76 of the single main channel 14, the embryo 12 is ready for vitrification, and the user may also use this single main channel 14 for vitrification by plunging it into a vitrification solution, such as liquid nitrogen.

While various embodiments of the present disclosure have been described, the present disclosure is not to be restricted except in light of the attached claims and their equivalents. One skilled in the relevant art will recognize that numerous variations and modifications may be made to the embodiments described above without departing from the scope of the present invention, as defined by the appended claims. Moreover, the advantages described herein are not necessarily the only advantages of the present disclosure and it is not necessarily expected that every embodiment of the present disclosure will achieve all of the advantages described.

We claim:

1. A system for passive permeation of a biological material, comprising:
a main channel extending between an upper portion and a lower portion;
a main reservoir connected to the upper portion of the main channel and in fluid communication with the main channel;
a bottom reservoir connected to the lower portion of the main channel and in fluid communication with the main channel;
at least one secondary channel disposed in at least one position between the upper portion and the lower portion of the main channel such that fluid communication is established in the at least one position between the at least one secondary channel and the main channel; and
at least one secondary reservoir respectively connected to an upper portion of each of the at least one secondary channel and in fluid communication with the respective at least one secondary channel.

2. The system for passive permeation of a biological material of claim 1,
wherein the main channel is configured such that a biological material placed in the main reservoir migrates by gravity along the main channel to the bottom reservoir, and
wherein the at least one secondary channel is configured to allow at least one secondary liquid placed in the at least one secondary reservoir to flow into the main channel and mix with a main liquid flowing from the main reservoir such that at least one mixed liquid is formed along a length of the main channel.

3. The system for passive permeation of a biological material of claim 2, wherein the at least one secondary liquid comprises at least one cryoprotectant solution.

4. The system for passive permeation of a biological material of claim 2, wherein the at least one secondary channel comprises a first secondary channel and a second secondary channel.

5. The system for passive permeation of a biological material of claim 4, wherein the at least one secondary liquid comprises a first secondary liquid and a second secondary liquid with different cryoprotectant concentrations, wherein the first and second secondary liquids are respectively disposed in the first and second secondary channels.

6. The system for passive permeation of a biological material of claim 5, wherein the first secondary liquid has a first cryoprotectant concentration, the second secondary liquid has a second cryoprotectant concentration, and the second cryoprotectant concentration is greater than the first cryoprotectant concentration.

7. The system for passive permeation of a biological material of claim 6, wherein the system is configured such that after allowing the first secondary liquid and the second secondary liquid to mix with the main liquid for respective predetermined amount of time, first, second, and third sections of the main channel with increasing cryoprotectant concentrations are respectively established, along the length of the main channel, between the main reservoir and the first secondary channel, between the first and second secondary channels, and between the second secondary channel and the bottom reservoir.

8. The system for passive permeation of a biological material of claim 7, wherein the system is configured such that the biological material migrating along the main channel sequentially travels through the first, second, and third sections for respective first, second, and third predetermined amount of time.

9. The system for passive permeation of a biological material of claim 8, wherein the first, second, and third predetermined amount of time are selected such that when the biological material migrating from the main reservoir reaches the bottom reservoir, the biological material is ready for vitrification.

10. The system for passive permeation of a biological material of claim 2, wherein the at least one secondary channel is configured such that the at least one secondary channel is at a higher pressure than the main channel such that the biological material migrates along the main channel towards the bottom reservoir without migrating towards the at least one secondary channel.

11. The system for passive permeation of a biological material of claim 2, wherein the biological material is an embryo or an oocyte.

12. The system for passive permeation of a biological material of claim 2, wherein the at least one secondary channel each comprise a valve automatically controlled for preventing the main liquid and the biological material from flowing into the at least one secondary channel.

13. The system for passive permeation of a biological material of claim 2, further comprising a port configured such that the biological material can be flushed out of the system.

14. The system for passive permeation of a biological material of claim 2, wherein an outer surface of the system is removable such that the biological material disposed in the system can be manually retrieved by a user.

15. The system for passive permeation of a biological material of claim 1, wherein the main channel is configured to accommodate a blastocyst-stage embryo.

16. The system for passive permeation of a biological material of claim 2, wherein the system is made of clear plastic such that a process of the biological material migrating from the main reservoir to the bottom reservoir is visible to a user.

17. The system for passive permeation of a biological material of claim 12, wherein the valve is a check valve.

18. A system for cryopreservation of a biological material, comprising:
a main channel extending between an upper end and a bottom end,
wherein the main channel comprises a first section disposed proximate to the upper end of the main channel and a second section disposed proximate to the bottom end of the main channel,
wherein the first section is filled with a first liquid having a first cryoprotectant concentration and the second section is filled with a second liquid having a second cryoprotectant concentration greater than the first cryoprotectant concentration,
and wherein the main channel is configured such that a biological material placed into the main channel through the upper end migrates towards the bottom end by gravity, and when the biological material reaches the bottom end, the biological material is ready for vitrification.

19. A method of passively permeating a biological material using a system comprising a main channel in fluid communication with at least one secondary channel, the main channel being connected to a main reservoir at an upper portion of the main channel and being connected to a bottom reservoir at a lower portion of the main channel, and the at least one secondary channel being connected to at least one secondary reservoir, comprising:
placing a first predetermined amount of a main liquid in the main reservoir;
placing at least one predetermined amount of at least one secondary liquid in the at least one secondary reservoir;
allowing the main liquid to flow along the main channel;
allowing the at least one secondary liquid to flow along the at least one secondary channel, and then into the main channel and mix with the main liquid to form at least one mixed liquid; and
placing a biological material in the main reservoir such that the biological material migrates along the main channel by gravity to travel through the at least one mixed liquid and reaches the bottom reservoir.

20. The method of passively permeating a biological material using a system of claim 19, wherein the at least one secondary liquid comprises at least one cryoprotectant solution.

21. The method of passively permeating a biological material using a system of claim 19, wherein the at least one secondary channel comprises a first secondary channel and a second secondary channel.

22. The method of passively permeating a biological material using a system of claim 21, wherein the at least one secondary liquid comprises a first secondary liquid and a second secondary liquid with different cryoprotectant concentrations, wherein the first and second secondary liquids are respectively placed in the first and second secondary channels.

23. The method of passively permeating a biological material using a system of claim 22, wherein the first secondary liquid has a first cryoprotectant concentration, the second secondary liquid has a second cryoprotectant concentration, and the second cryoprotectant concentration is greater than the first cryoprotectant concentration.

24. The method of passively permeating a biological material using a system of claim 23, wherein after allowing the first secondary liquid and the second secondary liquid to mix with the main liquid for respective predetermined amount of time, first, second, and third sections of the main channel with increasing cryoprotectant concentrations are respectively established, along a length of the main channel, between the main reservoir and the first secondary channel, between the first and second secondary channels, and between the second secondary channel and the bottom reservoir.

25. The method of passively permeating a biological material using a system of claim 24, the biological material migrating along the main channel sequentially travels through the first, second, and third sections for respective first, second, and third predetermined amount of time.

26. The method of passively permeating a biological material using a system of claim 25, wherein the first, second, and third predetermined amount of time are selected such that when the biological material migrating from the main reservoir reaches the bottom reservoir, the biological material is ready for vitrification.

27. The method of passively permeating a biological material using a system of claim 19, wherein the biological material is an embryo or an oocyte.

28. The method of passively permeating a biological material using a system of claim 19, wherein the at least one secondary channel is configured such that the at least one secondary channel is at a higher pressure than the main channel such that the biological material migrates along the main channel towards the bottom reservoir without migrating towards the at least one secondary channel.

29. The method of passively permeating a biological material using a system of claim 19, wherein the at least one secondary channel each comprise a valve automatically controlled for preventing the main liquid and the biological material from flowing into the at least one secondary channel.

30. The method of passively permeating a biological material using a system of claim 19, wherein the system comprises a port configured such that the biological material can be flushed out of the system.

31. The method of passively permeating a biological material using a system of claim 19, wherein an outer surface of the system is removable such that the biological material disposed in the system can be manually retrieved by a user.

32. The method of passively permeating a biological material using a system of claim 19, wherein the main channel of the system is configured to accommodate a blastocyst-stage embryo.

33. The method of passively permeating a biological material using a system of claim 19, wherein the system is made of clear plastic such that a process of the biological material migrating from the main reservoir to the bottom reservoir is visible to a user.

34. The method of passively permeating a biological material using a system of claim 29, wherein the valve is a check valve.
